(19) United States
(12) Patent Application Publication
(10) Pub. No.: US 2023/0139790 A1
(43) Pub. Date: May 4, 2023

(54) Neuromodulation of Primary and/or Postsynaptic Neurons

(71) Applicant: Saluda Medical Pty Ltd, Artarmon (AU)

(72) Inventor: Gerrit Eduard Gmel, Artarmon (AU)

(73) Assignee: Saluda Medical Pty Ltd, Artarmon (AU)

(21) Appl. No.: 17/759,364

(22) PCT Filed: Jan. 25, 2021
(86) PCT No.: PCT/AU2021/050043

§ 371 Date: Jul. 22, 2022

(30) Foreign Application Priority Data
Jan. 23, 2020 (AU) 2020900184

(51) Int. Cl. A61B 5/388

(52) U.S. Cl. CPC A61B 5/388; A61N 1/05

(57) ABSTRACT

A neurostimulation system comprises at least one stimulation electrode configured to deliver an electrical stimulus to neural tissue and at least one measurement electrode configured to record a neural recording of a response of the neural tissue to the stimulus. A processor is configured to assess the neural recording to produce a measure of postsynaptic activation.

### CROSS-REFERENCE TO RELATED APPLICATIONS

This application claims the benefit of Australian Provisional Patent Application No. 2020900184 filed 23 Jan. 2020, which is incorporated herein by reference.

### TECHNICAL FIELD

The present invention relates to methods and devices for neurostimulation, and in particular to a method and device for assessing recruitment of primary and/or postsynaptic neurons from electrophysiological response measurements.

### BACKGROUND OF THE INVENTION

There are a range of situations in which it is desirable to apply neural stimuli in order to give rise to a compound action potential (CAP). For example, neuromodulation is used to treat a variety of disorders including chronic pain, Parkinson's disease, and migraine. A neuromodulation system applies an electrical pulse to tissue in order to generate a therapeutic effect. When used to relieve chronic pain, the electrical pulse is typically applied to the dorsal column (DC) of the spinal cord, referred to as spinal cord stimulation (SCS). Neuromodulation systems typically comprise an implanted electrical pulse generator, and a power source such as a battery that may be rechargeable by transcutaneous inductive transfer. An electrode array is connected to the pulse generator, and is positioned in the dorsal epidural space above the dorsal column. An electrical pulse applied to the dorsal column by an electrode causes the depolarisation of neurons, and generation of propagating action potentials. To sustain the pain relief effects, stimuli are applied substantially continuously, for example at a frequency in the range of 50-100 Hz.

Neuromodulation may also be used to stimulate efferent fibres, for example to induce motor functions. In general, the electrical stimulus generated in a neuromodulation system triggers a neural action potential which then has either an inhibitory or excitatory effect. Inhibitory effects can be used to modulate an undesired process such as the transmission of pain, or to cause a desired effect such as the contraction of a muscle.

While the clinical effect of spinal cord stimulation (SCS) is well established, the precise mechanisms involved are poorly understood.

There are a range of circumstances in which it is desirable to obtain an electrical measurement of an evoked compound action potential (ECAP) evoked on a neural pathway by an electrical stimulus applied to the neural pathway. However, in practical implantable devices this can be a difficult task as an observed ECAP signal will typically have a maximum amplitude of a few tens of microvolts or less, whereas a stimulus applied to evoke the ECAP is typically several volts.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

In this specification, a statement that an element may be “at least one of” a list of options is to be understood that the element may be any one of the listed options, or may be any combination of two or more of the listed options.

### SUMMARY OF THE INVENTION

According to a first aspect the present invention provides a neurostimulation system comprising:

at least one stimulation electrode configured to deliver an electrical stimulus to neural tissue;

at least one measurement electrode configured to record a neural recording of a response of the neural tissue to the stimulus; and

a processor configured to assess the neural recording to produce a measure of postsynaptic activation.

According to a second aspect the present invention provides a method of neurostimulation, the method comprising:

delivering an electrical stimulus to neural tissue using at least one stimulation electrode;

obtaining a neural recording of a response of the neural tissue to the stimulus using at least one measurement electrode, and

assessing the neural recording to produce a measure of postsynaptic activation.

According to a third aspect the present invention provides a non-transitory computer readable medium for neurostimulation, comprising instructions which, when executed by one or more processors, causes performance of the following: delivering an electrical stimulus to neural tissue using at least one stimulation electrode;

obtaining a neural recording of a response of the neural tissue to the stimulus using at least one measurement electrode, and

assessing the neural recording to produce a measure of postsynaptic activation.

Some embodiments of the present invention advantageously provide an ability to separately assess primary neuron activation as a result of a stimulus as compared to postsynaptic activation resulting from the stimulus, and may further differentiate between axonal activation of postsynaptic fibres and synaptic activation of postsynaptic fibres. Depending on a relevant disease state which may be present, or a therapeutic mode of activation which is clinically desired, the relative levels of activation of primary neurons vs. postsynaptic neurons and/or the relative levels of axonal vs synaptic activation of postsynaptic fibres may thus provide an important diagnostic tool and/or therapy optimisation tool.

It is to be appreciated that post-synaptic neurons may be activated either directly by applied electrical stimulation, also referred to herein as axonal activation, or may be activated by natural synapsing from primary neurons, also referred to herein as synaptic activation. Further, references herein to “presynaptic” fibres or neurons may refer to primary fibres which branch and which have a branch extending to a synapse but which also have a branch extending beyond the synapse, and it is to be understood herein that references to presynaptic fibres encompass such branching primary fibres.

The neural tissue may comprise any neural pathway or fibre tract comprising both presynaptic and postsynaptic fibres. Along such a pathway, the presynaptic fibres have a monosynaptic or polysynaptic connection onto the postsynaptic fibres such that the postsynaptic fibres can, under the right circumstances, be activated by activity in the presynaptic fibres. For example, in some embodiments, the neural tissue may comprise the spinal cord, for example in the lumbar enlargement, thorax, and/or the cervical enlargement, and the neural pathway may comprise sensory primary afferents alongside postsynaptic dorsal column fibres. In some embodiments the postsynaptic fibres alongside the presynaptic fibres are activated synaptically by action potentials in the presynaptic fibres. Similarly, “primary afferent” as used herein typically refers to mechanoreceptors, however the present invention also encompasses situations arising from any synapse where the postsynaptic fibres run alongside the “primary” fibres in a common fibre tract observable by measurement electrodes.

The system may comprise an implantable neurostimulator. Alternatively, the system may be partly non-implanted, for example the processor may be non-implanted and may comprise an external component for use in a surgical or clinical review setting.

In some embodiments, the measure of postsynaptic activation may be produced by assessing a single neural recording, and inspecting the recording for additional lobes, such as by identifying a number of lobes of a neural response observed in the neural recording and/or a magnitude of each identified lobe. For example, where a characteristic CAP is expected to comprise three lobes (e.g., P1, N1 and P2 lobes) an amplitude of a fourth peak (e.g. an N2 peak) and/or an amplitude of a fifth peak (e.g. a P3 peak) may be relied upon to produce the measure of postsynaptic activation. A presence or strength of such additional lobes may be used as a measure of the presence or strength of synaptic activation of postsynaptic fibres, which may in turn be used for example to assess a state of the intervening synapse(s) such as whether such synapse(s) is/are normal or abnormal, or has/have changed over time. In such embodiments the at least one stimulation electrode preferably delivers the stimulus at a vertebral level below T8, more preferably below T10, most preferably below T12. In such embodiments, the at least one measurement electrode is preferably positioned orthodromically of the stimulus electrode.

Alternatively, in some embodiments, a plurality of stimuli are applied, and a plurality of neural recordings of a response of the neural tissue to the respective stimulus are obtained from the at least one measurement electrode, and a plurality of measures of postsynaptic activation in each respective neural recording are obtained. The plurality of neural recordings may additionally or alternatively be obtained from a plurality of measurement electrodes positioned along the fibre tract, such as being positioned caudorostrally alongside the dorsal column, to allow a conduction velocity of an evoked neural response and/or conduction velocities of two or more components of an evoked neural response to be ascertained by comparing a time of arrival of neural recordings obtained at different measurement electrodes. In such embodiments, where it is determined that a single observed evoked compound activation potential (ECAP) comprises at least two components conducting at different conduction velocities, it may be concluded that the observed ECAP is a result of activation of two different fibre types and that each fibre type has been activated axonally and not synaptically due to the absence of a synaptic delay temporally separating the two components. For example, in the particular case of SCS, an observed conduction velocity of 90 m/s can be concluded as corresponding to axonal activation of postsynaptic fibres only, and thus the associated stimulation paradigm may not optimally effect pain relief, as it is understood that activation of primary afferents is desired to effect pain relief. Primary afferents have a typical conduction velocity of about 45 m/s and so an absence of any such observed component in the neural response can be taken to indicate an absence of activation of primary afferents, and that the activation of postsynaptic fibres must be axonal and not synaptic. This insight allows for a fitting process to optimise primary afferent activation by revising any suitable stimulation parameter so as to seek a desired degree of activation of primary afferents and/or postsynaptic fibres, such as by revising a choice of stimulus electrode location or number of stimulus electrodes (bipolar, tripolar, etc), and/or by revising electrical parameters such as stimulus amplitude, duration, pulse width, number of pulses (biphasic, triphasic, etc) and the like. In some cases it may be that having a mix of activation is optimal, and such embodiments may provide for the stimulation paradigm to be iteratively revised so as to seek a conduction velocity greater than 45 m/s and less than 90 m/s, such as about 60 m/s, so that a similar or equal number of primary afferents and postsynaptic fibres are activated.

In some embodiments, the measures of postsynaptic activation may comprise a binary output indicating only whether or not postsynaptic activation is present. Alternatively, the measures of postsynaptic activation may be gradated in order to indicate a magnitude of observed postsynaptic activation.

In some embodiments, the presence, absence or magnitude of the measures of postsynaptic activation may be used as a diagnostic tool to diagnose dorsal horn sensitisation. For example, in some cases high postsynaptic activation may be taken to indicate dorsal horn sensitisation. Alternatively, synaptic desensitisation may occur in some cases in which case a reduction in, absence of or low level of postsynaptic activation may enable a diagnosis of desensitisation. Thus a relative or absolute measure of synaptic strength may be enabled in some embodiments of the invention and may permit diagnosis.

In some embodiments the presence or absence or magnitude of the measures of postsynaptic activation may be obtained repeatedly when applying spinal cord stimulation at different vertebral levels, and may be used to determine which vertebral levels are affected by synaptic sensitisation/desensitisation, and/or such measures may be used to assist electrode selection or positioning or to assist optimisation of stimulation parameters relative to the synaptic information represented in the measures of postsynaptic activation. In general, the measures of postsynaptic activation may be used as a diagnostic tool to determine the state of the patient with respect to the synaptic strength for any pathway with a synapse and postsynaptic fibres. This can also be a proxy measure for synaptic sensitisation/desensitisation in general. Some embodiments may use a determination of synaptic sensitivity at one or more vertebral levels as an input a diagnostic tool to predict whether a given patient will respond well to SCS. Some embodiments may obtain measures of synaptic state over time in order to identify a change in synaptic state, for example as may occur upon intake, weaning or cessation of medication, or as may occur in response to neuromodulation. In such embodiments, a further step may occur of changing stimulation parameters in order to avoid or reduce stimulation of fibres which cause undesirable synaptic activation, or in order to target or increase stimulation of fibres which cause desirable synaptic activation.

In some embodiments of the invention, the one or more measures of postsynaptic activation may be used to revise the stimulation parameters of a subsequent stimulus, for example to alter a proportion of postsynaptic activation relative to primary afferent activation. In such embodiments, the stimulation parameters may be revised in order to seek an increased amount of postsynaptic activation relative to dorsal column activation, or a decreased amount such as no postsynaptic activation. As some modes of stimulation might be particularly inhibitory or excitatory for the synapse, such stimulation parameters which may be optimised could include any or all of burst mode, stimulation rate (e.g. 40 Hz vs 10 Hz), escalators (as described in WO2012155187), and the like.

In some embodiments, neural recordings of the or each evoked response may be obtained from at least one recording electrode positioned orthodromically from the stimulus site, and from at least one recording electrode positioned antidromically from the stimulus site. In such embodiments assessing the neural recordings to identify postsynaptic activation may comprise assessing whether orthodromic postsynaptic activation exceeds antidromic postsynaptic activation, noting that antidromic postsynaptic activation is typically absent, for example to improve accuracy of identification of neural response components as truly postsynaptic. Alternative embodiments may nevertheless achieve suitable efficacy by only using recording electrodes which are positioned orthodromically of the stimulus electrodes.

In some embodiments, assessing the neural recordings may comprise identifying postsynaptic activation by determining whether a synaptic delay is present in the onset of the respective neural response component. For example, a synaptic delay may be determined by identifying at least two components of neural activation in each of a plurality of neural recordings, assessing propagation of each component past the respective recording electrodes, determining a time of origin of each component, and determining whether origination of one component is delayed relative to the other, such as by an amount of time consistent with synaptic delay. For example a time of origin of each component may be determined by identifying for each component a respective y-intercept of a respective line fitted to a plot of time and distance observations of each component.

In some embodiments of the invention, the measures of neural activation comprise ECAP amplitude measures, for example a measure of N1-P2 peak to peak amplitude for a first component of activation, and a measure of N2-P3 peak to peak amplitude for a second component of activation.

The neural recordings may in some embodiments be obtained in accordance with the teachings of the present Applicant for example in U.S. Pat. No. 9,386,934, International Patent Application No. PCT/AU2019/051151, International Patent Application No. PCT/AU2019/051160, and/or International Patent Application No. PCT/AU2019/051385 the content of each of which are incorporated herein by reference. The conduction velocity of the or each neural component may in some embodiments be determined in accordance with the teachings of International Patent Application No. PCT/AU2019/051197, the content of which is incorporated herein by reference.

In further embodiments of the invention the neural recording and/or measure of postsynaptic activation may comprise any of the above noted measures, compensated for a distance-dependent transfer function of stimulation, and/or compensated for a distance dependent transfer function of measurement. Such distance dependent transfer function compensation may be implemented in the manner described in the present Applicant's International Patent Publication No. WO2017173493, the content of which is incorporated herein by reference. The neural recording and/or measure of postsynaptic activation in some embodiments may be normalised to compensate for variations caused by postural changes, or physiological events such as coughs, heartbeat and breathing. Component-specific normalisation may also be appropriate in order to compensate for non-parallel paths of presynaptic and postsynaptic neurons along the dorsal column.

Some embodiments may further provide for assessing the neural recording to produce a measure of presynaptic activation, such as a measure of activation of primary sensory afferents. The measure of presynaptic activation may be produced by assessing the neural recording to ascertain an amplitude of a second peak (e.g. an N1 peak) and/or an amplitude of a third peak (e.g. a P2 peak), to produce the measure of presynaptic activation.

The identification of postsynaptic activation in some embodiments may be used to optimise therapy by guiding changes to selection of the stimulating electrode(s). The identification of postsynaptic activation in some embodiments may be used to optimise therapy by guiding changes to selection of recording electrode(s). The identification of postsynaptic activation in some embodiments may be used to optimise therapy by guiding changes to selection of a combination of recording and stimulating electrodes. The identification of postsynaptic activation in some embodiments may be used to optimise therapy by guiding changes to selection of stimulus parameters including a stimulus intensity, selection of a stimulus current, selection of stimulus pulse width(s), selection of stimulus frequency, and/or selection of stimulus pulse shape. The identification of postsynaptic activation in some embodiments may be used to optimise therapy by optimising paraesthesia as reported by the implantee. The identification of postsynaptic activation in some embodiments may be used to optimise therapy by guiding changes to selection of measurement amplifier settings. The identification of postsynaptic activation in some embodiments may be used to optimise therapy by guiding changes to selection of a target level of neural activation. The identification of postsynaptic activation in some embodiments may be used to optimise therapy by guiding changes to selection of feedback loop implementation such as selection among feedback loop implementations set out in WO2017173493. The identification of postsynaptic activation in some embodiments may be used to optimise therapy by guiding changes to selection of feedback loop parameters, such as feedback loop gain, feedback loop noise bandwidth, and feedback loop instant backoff threshold. The identification of postsynaptic activation in some embodiments may be used to optimise therapy by guiding changes to a degree of synchronisation or desynchronization of primary activation and postsynaptic activation.

Further embodiments of the invention may comprise an automated procedure for improving therapeutic efficacy, by iteratively revising the therapy to seek an improvement in the identification of postsynaptic activation and/or an improvement in the activation or inactivation of the synaptic transmission. Any suitable systematic method of revising patient settings that improved the resulting observed postsynaptic activation may be selected. For example, a feedback loop gain may be optimised by an iterative process involving: (i) measuring a first postsynaptic activation with feedback loop gain set at a first value, (ii) adjusting feedback loop gain from a first value to a second value, (iii) measuring a second postsynaptic activation and (iv) if the second postsynaptic activation indicates higher therapeutic efficacy than the first postsynaptic activation, retaining the second value of loop gain for ongoing use. Such an iterative procedure may be repeated any number of times required to sufficiently explore the available range of options for the feedback loop gain and to find an optimal value. A corresponding iterative procedure may be performed in respect of any aspect of operation of the device.

In some embodiments of the invention, the measurement circuitry is configured to obtain the recordings of the neural responses substantially continuously during device operation. For example, in some embodiments of the invention the implanted neuromodulation device is configured to record the recordings of the neural responses for a period of at least 8 hours of device operation. In some embodiments of the invention the implanted neuromodulation device is configured to record the recordings of the neural responses for a period of at least 2 days of device operation. In some embodiments of the invention the implanted neuromodulation device is configured to record the recordings of the neural responses for a period of at least 5 days of device operation. To this end, preferred embodiments of the invention provide for the implanted neuromodulation device to be configured to process each recording of a neural response in substantially real time in order to obtain a respective measure of postsynaptic activation, and further provide for the implanted neuromodulation device to store in memory only the measure of postsynaptic activation and not the entire recording. For example, the implanted neuromodulation device may store in memory a histogram of the plurality of measures of postsynaptic activation in the form of a plurality of bins, with a counter associated with a respective bin being incremented each time an additional measure of postsynaptic activation is obtained. Such embodiments permit such data to be obtained over a period of hours or days at a high rate, such as at 50 Hz or more, and to be stored in very compact manner by use of a histogram and to thereby avoid exceeding the limited memory constraints of an implantable device. The bins may each be allocated a width, or range, which is equal for each bin. Alternatively, the bins may be allocated respective widths which increase with increasing levels of postsynaptic activation, such as linearly increasing bin widths or exponentially increasing bin widths.

References herein to estimation, determination, comparison and the like are to be understood as referring to an automated process carried out on data by a processor operating to execute a predefined procedure suitable to effect the described estimation, determination and/or comparison step(s). The approaches presented herein may be implemented in hardware (e.g., using application specific integrated circuits (ASICS)), or in software (e.g., using instructions tangibly stored on computer-readable media for causing a data processing system to perform the steps described herein), or in a combination of hardware and software. The invention can also be embodied as computer-readable code on a computer-readable medium. The computer-readable medium can include any data storage device that can store data which can thereafter be read by a computer system. Examples of the computer readable medium include read-only memory (“ROM”), random-access memory (“RAM”), CD-ROMs, DVDs, magnetic tape, optical data storage device, flash storage devices, or any other suitable storage devices. The computer-readable medium can also be distributed over network coupled computer systems so that the computer readable code is stored and executed in a distributed fashion.

According to a further aspect the present invention provides a method of treating a neural disease, the method comprising:

ordering or requesting the result of the method of the second aspect; and

administering or modifying a therapy in a manner responsive to the ordered result.

### BRIEF DESCRIPTION OF THE DRAWINGS

An example of the invention will now be described with reference to the accompanying drawings, in which:

FIG. 1 schematically illustrates an implanted spinal cord stimulator;

FIG. 2 is a block diagram of the implanted neurostimulator;

FIG. 3 is a schematic illustrating interaction of the implanted stimulator with a nerve;

FIG. 4 illustrates a typical SCS electrode lead implantation arrangement;

FIG. 5 is a plot of two neural recordings from different patients;

FIG. 6 illustrates ECAP component propagation along the electrode array for both patients;

FIG. 7 is a plot of neural recordings of a single neural response obtained by using a plurality of recording electrodes along the lead;

FIG. 8 is a plot of recordings made with large lead migration;

FIG. 9 is a neural schematic illustrating presynaptic and postsynaptic neurons; and

FIG. 10 is a further neural schematic illustrating presynaptic and postsynaptic neurons.

### DESCRIPTION OF THE PREFERRED EMBODIMENTS

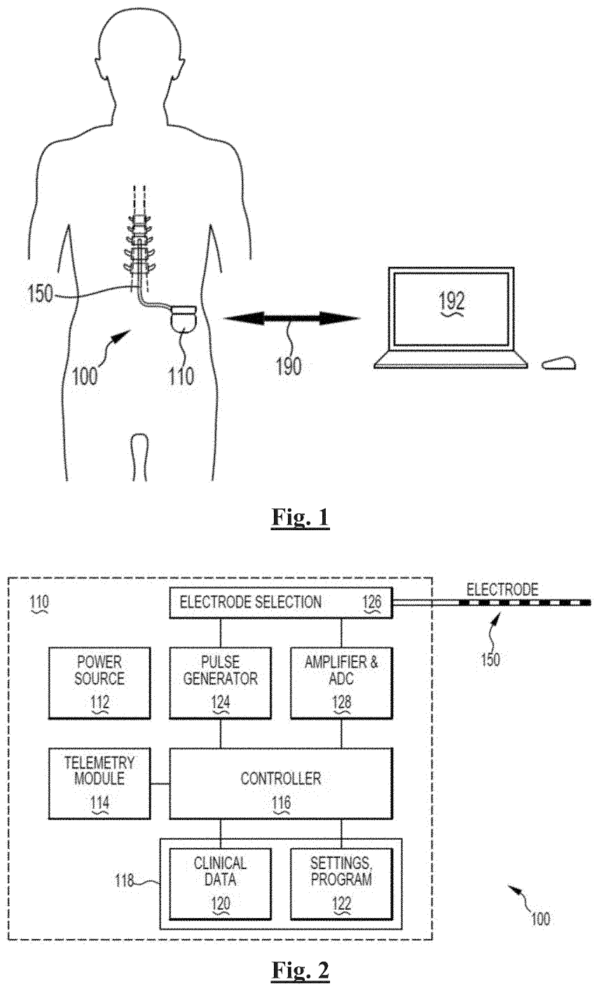

FIG. 1 schematically illustrates an implanted spinal cord stimulator 100. Stimulator 100 comprises an electronics module 110 implanted at a suitable location in the patient's lower abdominal area or posterior superior gluteal region, and an electrode assembly 150 implanted within the epidural space and connected to the module 110 by a suitable lead. Numerous aspects of operation of implanted neural device 100 are reconfigurable by an external control device 192. Moreover, implanted neural device 100 serves a data gathering role, with gathered data being communicated to external device 192 via any suitable transcutaneous communications channel 190.

FIG. 2 is a block diagram of the implanted neurostimulator 100. Module 110 contains a battery 112 and a telemetry module 114. In embodiments of the present invention, any suitable type of transcutaneous communication 190, such as infrared (IR), electromagnetic, capacitive and inductive transfer, may be used by telemetry module 114 to transfer power and/or data between an external device 192 and the electronics module 110. Module controller 116 has an associated memory 118 storing patient settings 120, control programs 122 and the like. Controller 116 controls a pulse generator 124 to generate stimuli in the form of current pulses in accordance with the patient settings 120 and control programs 122. Electrode selection module 126 switches the generated pulses to the appropriate electrode(s) of electrode array 150, for delivery of the current pulse to the tissue surrounding the selected electrode(s). Measurement circuitry 128 is configured to capture measurements of neural responses sensed at sense electrode(s) of the electrode array as selected by electrode selection module 126.

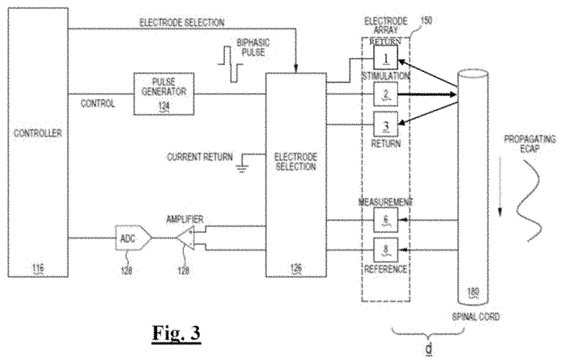

FIG. 3 is a schematic illustrating interaction of the implanted stimulator 100 with a nerve 180, in this case the spinal cord however alternative embodiments may be positioned adjacent any desired neural tissue including a peripheral nerve, visceral nerve, parasympathetic nerve or a brain structure. Electrode selection module 126 selects a stimulation electrode 2 of electrode array 150 to deliver an electrical current pulse to surrounding tissue including nerve 180, and also selects a return electrode 4 of the array 150 for stimulus current recovery to maintain a zero net charge transfer.

Delivery of an appropriate stimulus to the nerve 180 evokes a neural response comprising a compound action potential which will propagate along the nerve 180 as illustrated, for therapeutic purposes which in the case of a spinal cord stimulator for chronic pain might be to create paraesthesia at a desired location. To this end the stimulus electrodes are used to deliver stimuli at any therapeutically suitable frequency, for example 30 Hz, although other frequencies may be used including as high as the kHz range, and/or stimuli may be delivered in a non-periodic manner such as in bursts, or sporadically, as appropriate for the patient. To fit the device, a clinician applies stimuli of various configurations which seek to produce a sensation that is experienced by the user as a paraesthesia. When a stimulus configuration is found which evokes paraesthesia, which is in a location and of a size which is congruent with the area of the user's body affected by pain, the clinician nominates that configuration for ongoing use.

The device 100 is further configured to sense the existence and intensity of compound action potentials (CAPs) propagating along nerve 180, whether such CAPs are evoked by the stimulus from electrodes 2 and 4, or otherwise evoked. To this end, any electrodes of the array 150 may be selected by the electrode selection module 126 to serve as measurement electrode 6 and measurement reference electrode 8. The reference electrode could alternatively be located on the case of the device or externally. Signals sensed by the measurement electrodes 6 and 8 are passed to measurement circuitry 128, which for example may operate in accordance with the teachings of International Patent Application Publication No. WO2012155183 by the present applicant, the content of which is incorporated herein by reference.

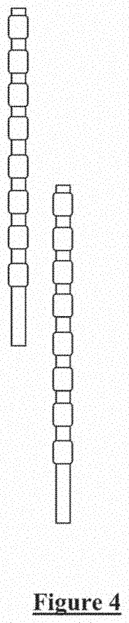

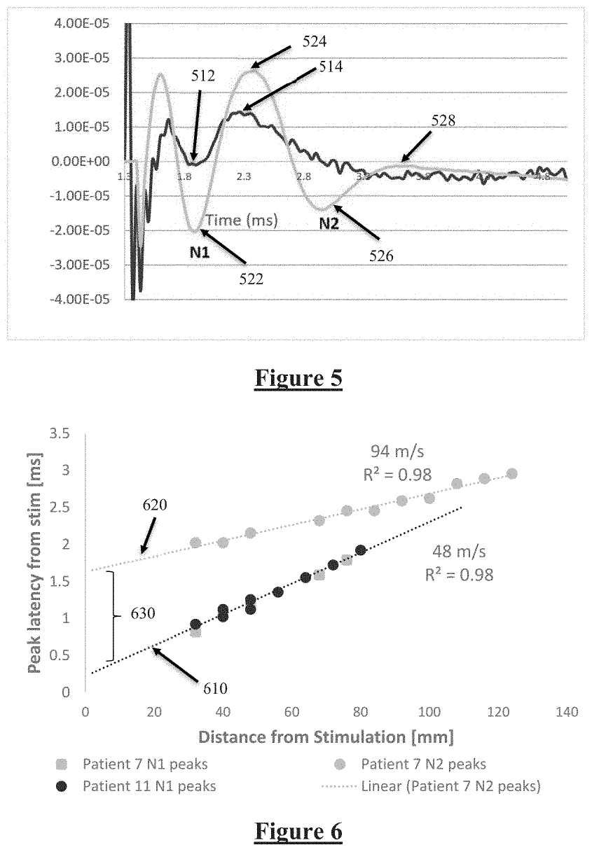

In a study conducted by the Applicant, 16 patients undergoing SCS for neuropathic pain in the lower back and/or legs were recruited to participate. Two 8-contact leads (8 mm inter-electrode spacing) were implanted according to standard practice with an overlap of 2-4 contacts around the T9/T10 interspace. FIG. 4 illustrates this typical lead arrangement: two 8-contact leads (8 mm centre-centre pitch) with a standard overlap of 3 contacts around T9/T10 interspace. During two routine follow-up visit in the trial period, we carried out current and frequency sweeps on various locations on the leads using a custom external stimulator capable of real-time ECAP recording from all non-stimulating contacts. FIG. 5 illustrates an example of a first neural recording containing a 3-lobe ECAP (Patient 07), with the N1 peak 512 and P2 peak 514 indicated, while peak P1 preceding peak N1 is partly obscured. FIG. 5 also contains a plot of a second neural recording containing a 5-lobe ECAP (Patient 11), exhibiting an N1 peak 522, P2 peak 524, N2 peak 526 and P3 peak 528.

The present embodiment recognises that it is possible to follow the N1 and N2 peaks as separate observations, or separate components of the neural response, as they propagate along the nerve alongside the electrode lead. FIG. 6 illustrates N1 peak propagation along the electrode array for both patients 07 and 11. A single line 610 can be fitted to these N1 observations, from both of the two patients. FIG. 6 further illustrates N2 peak propagation along the electrode array for patient 7, noting that patient 11 did not exhibit any N2 peaks. A line 620 can be fitted to the N2 observations. The different slope of line 610 as compared to the slope of line 620 reveals that the conduction velocity of the N2 peak is distinct from, and faster than, the conduction velocity of the N1 peak. As indicated at 630, it can also be deduced that the N2 peak originates about lms after the first one, from which it can be deduced that the N2 peak is part of a different second ECAP which is evoked at a different time to the first ECAP contributing the N1 peak. In this case the second ECAP contributing the N2 peak propagates at about 94 m/s, about twice as fast as the 48 m/s velocity of the first ECAP. This relationship is somewhat fluid depending on the patient and other variables, however the second ECAP and in particular the N2 peak 526 has been observed to propagate faster than the N1 peak 522 in all experiments seen so far, making this a reliable means by which to separately assess separate components of a single ECAP observed along multiple recording electrodes in this manner.

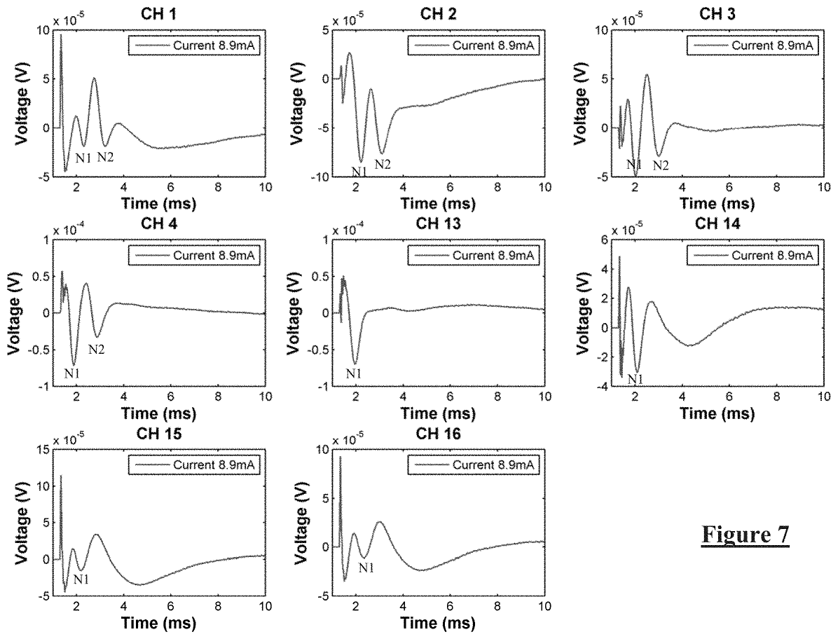

FIG. 7 is a plot of neural recordings of a single neural response, the recordings of that single neural response being obtained by using a plurality of recording electrodes along the lead. Stimulation was applied using a narrow tripole around electrode 7, which utilised the bottom electrodes of the top lead which were located around the T9/T10 interspace. Notably, a second response component in the form of a N2 peak and a P3 peak was only observed orthodromically, upon channels 1-4, i.e. rostrally for afferents. No second response component is observed antidromically upon channels 13-16. In this regard it is noted that CH13 clearly exhibits only a single N1 peak, found at time 2 ms on the recording, and no N2 peak. As the response propagated antidromically beyond electrode 13 to electrodes 14, 15 and 16, the respective recordings show that the N1 peak progressively arises later in time in each respective recording, arising at about 2.5 ms in the CH16 recording. From this it is important to note that the signal artefacts arising before 2 ms in the CH14-16 recordings can be disregarded in this particular experimental setup. The present inventors have thus noted that this second response component (N2 in FIG. 7) only occurs orthodromically, never antidromically. Thus, the second response component (second ECAP, peaks N2 and P3) is only observed on the electrodes higher than the stimulating electrodes while on the electrodes lower than the site of stimulation a single 3-lobe ECAP is observed. Accordingly, this characteristic also presents another means by which to separately assess separate components of a single ECAP observed along multiple recording electrodes in this manner. Moreover, this observation allows it to be deduced that the second response component is in particular a postsynaptic neural response, due at least to its one-directional propagating nature, and the temporal delay in onset caused by synaptic delay.

The activation of postsynaptic fibres may be activated by the first signal or potentially may be activated from the stimulation but requiring synaptic transmission.

In the studied cohort, over 50% of patients presented a 5 lobe ECAP when stimulation was applied at the bottom (most caudal end) of the lead. For most patients, as the site of stimulation was moved more rostrally, the 5 lobe ECAP no longer appears once stimulation is occurring around the middle of the bottom (rostral) lead. However, one patient exhibited a 5 lobe ECAP at all sites, even when stimulating at the top (rostral end) of the array. Without intending to be limited by theory, this may for example be indicative of that patient having very low synapse threshold which may in turn be indicative of a central sensitisation disease state. Irrespective of mechanism, this characteristic presents another means by which to separately assess, diagnose or treat individual patients.

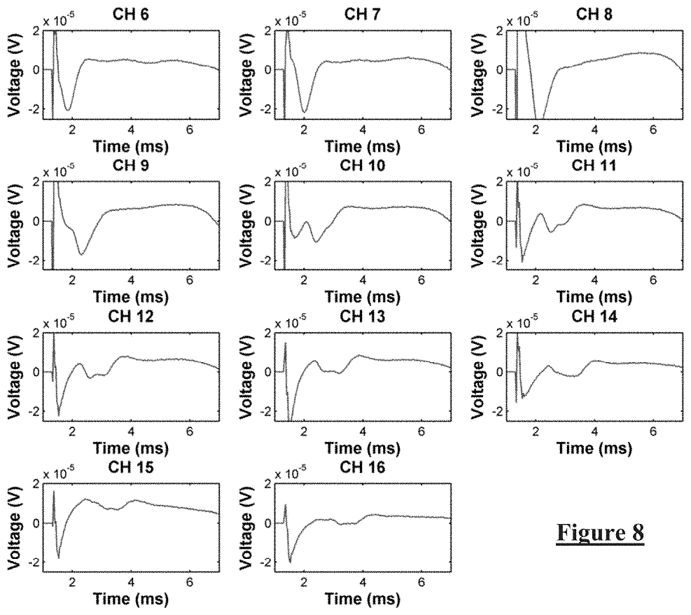

FIG. 8 is a plot of recordings made for Patient 07 with large lead migration. This illustrates that when stimulating on the top of the array, separation can be seen antidromically when the distance between the stimulation and recording is sufficient. In some instances, signal splitting can be seen at the bottom of the array (far away from the stimulation). This demonstrates that even when a single-lobe ECAP is observed at one measurement electrode, it may nevertheless be made up of the response of 2 different fibre populations, as is proven by the fact that when sufficient distance is achieved between stim and recording, the 2 populations “split”.

Current sweeps also confirmed a linear relationship between ECAP amplitude and paraesthesia sensation at constant frequency. Frequency sweeps showed an inverse relationship between stimulation sensation strength and ECAP amplitude, meaning that higher frequencies led to smaller ECAPs but higher stimulation sensation for constant stimulation amplitude.

The results support that stimulation sensation is conveyed through both frequency coding and population coding, fitting known psychophysics of tactile sensory information processing. However, the present invention recognises that the inverse relationship between ECAP amplitude and sensation at increasing frequencies opens a new line of inquiry to investigate the relationship of neural activation and stimulus sensation.

From the preceding, we can deduce or postulate that the differences in ECAP morphology and conduction velocity obtained at different vertebral levels can be attributed to the activation of post-synaptic dorsal column (PSDC) fibres. The PSDC pathway has been widely ignored by the field of pain management and its link to sensation and pain relief presents an opportunity to improve diagnosis and therapy.

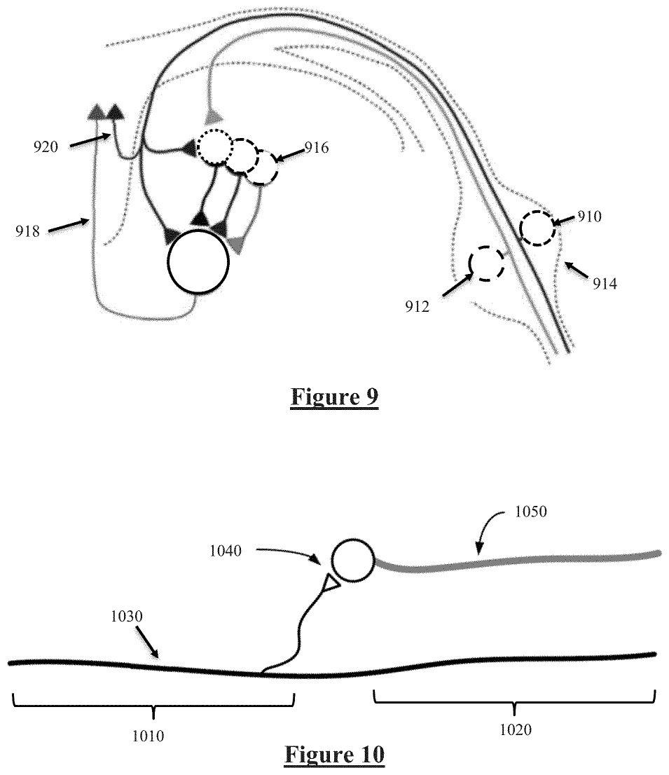

FIG. 9 is a neural schematic illustrating this situation, showing Aβ low threshold mechanoreceptors 910, C/Aδ low threshold mechanoreceptors 912, dorsal root ganglion 914, low threshold mechanoreceptors RZ interneurons 916, and postsynaptic neuron 918. It can be seen that both presynaptic afferents 920 and postsynaptic neuron(s) 918 project to the brain along a common fibre tract, and may both contribute to electrode recordings obtained orthodromically from the activation site, and moreover that activated primary afferents may activate postsynaptic neurons, as surmised in the preceding.

Postsynaptic dorsal column fibres known to exist may for example transmit cutaneous sensory information (most nuclei in laminae III and IV) and visceral sensory information (nuclei in lamina X). Selectively activating or selectively avoiding activating such fibres by way of neurostimulation in accordance with the present embodiment may thus provide a means by which to selectively apply therapy to such pathways.

It is further observed that a majority of primary afferents terminate and don't ascend all the way to the dorsal column nuclei. Postsynaptic dorsal column fibres on the other hand are likely to almost all ascend to the DC nuclei. Accordingly, selectively or preferentially activating one of these groups and not the other is likely to lead to therapeutic advantages for patients and may be individualised to each patient according to need.

In sum, the present inventors have observed ECAP morphologies when stimulating in some locations along the spinal cord that appear to be composed of two ECAPs, which in some locations appears as a “5-lobe ECAP” instead of the classic 3-lobe ECAP. Moreover, in many cases the second ECAP propagates faster than the first peak. The present embodiment in particular recognises that this characteristic of an observed neural response (comprising two or more response components, with the later-appearing component propagating more quickly in the case of the dorsal columns), allows such an observed response to be differentiated from a fast and slow ECAP being elicited at the same time, or even multiple ECAPs being elicited from burst firing of axons. In synapses other than those in the dorsal columns, the later-appearing (post-synaptic) component could have the same velocity or may be faster or slower than the first peak, however these features once observed nevertheless permit an assessment of post-synaptic activation to be made in a corresponding manner as described for the dorsal columns. Further, the difference between antidromic and orthodromic propagation discredits the idea of axonal damage as damaged axons would likely behave the same way in either direction.

Further embodiments are described below based on these findings. The doublets are made up of signals from primary afferents (PAs, the “first” ECAP) and postsynaptic dorsal column (PSDC) fibres (the 2nd ECAP). PSDC fibre are an integral part of the sensory system and the ones elicited are most likely sensory fibres from the skin and not the viscera (which have their nuclei in a much lower lamina of the spinal cord than the cutaneous sensory PSDC fibres). These PSDC nuclei are prevalent in the lumbar enlargement, which could explain why we see them most in the bottom parts of the lead.

It seems that the PSDC fibres ascend to the dorsal columns (from their dorsal horn nucleus) over a few millimetres, meaning that at lower levels, the ECAP is comprised only of PAs and travels at 45 m/s. This ECAP becomes smaller the further away it is from the stimulation site due to termination of fibres.

When a second population is elicited synaptically, they travel at faster speeds, say around 75-90+ m/s and are elicited about lms after the PA due to synapse delay.

Higher up (more rostrally) in the cord, the CV of the ECAP is around 60 m/s, this would indicate that there is a mix of slow (PA) and fast (PSDC) fibres rather than having one population of fibres travelling at 60 m/s. This is quite fundamental as it means that the ratio of PAs vs PSDC (and potentially other fibres such as descending inhibitory fibres and proprioceptive fibres) can be assessed by assessing the CV of the ECAP generated in the case where no synaptic activation is elicited. Proof of this can be seen in the example from Patient 07 where splitting of the ECAP can be seen at some distance away from the stimulation. This is consistent with 2 populations of fibres with distinct centre conduction velocities being activated at the same time and propagating way from their stimulus site; the longer the distance, the more clearly the 2 populations will be separated.

It is to be noted that PSDC fibres can be activated axonically or synaptically by spinal cord stimulation. So synaptic delay is not always present, only when the population of PSDC fibres has been activated by PAs via the synapse in the DH.

This knowledge leads to several applications discussed below, all of which revolve around the idea that the 5 lobe ECAPs seen are primary afferents (PAs) and postsynaptic dorsal column (PSDC) fibres. This is somewhat limiting in the case where no synaptic activation is observed. Obtaining an ECAP with 60 m/s CV could show a mix of fibres, but the nature of these fibres is somewhat unclear.

With regard to sensitisation it is notable that not all patients have synaptic PSDC activation when stimulating at the lower levels of the array. It is possible that the difference is purely anatomical, but it is likely that this shows a certain state of sensitisation of the PSDC pathway in particular, and potentially of the DH in general. Therefore, in one embodiment, the presence of synaptically activated PSDC fibres (similar to the example in FIG. 2) can be used as a diagnostic tool for DH sensitisation.

There is a chance this is linked to pain relief (as in that neuropathic pain is caused by sensitisation, or desensitisation of the DH, either this PSDC pathway directly, or via the classic pain pathway (or both)), and that therefore the presence or absence of doublets can indicate the aetiology of the patient's pain condition. Therefore this could be used to inform whether SCS should be used to treat the condition or not, or whether another or a complementary therapy should be employed.

Further, the presence or absence of synaptically activated ECAP components at different vertebral levels could be ascertained in order to: (i) indicate which vertebral levels are affected by sensitisation/desensitisation, (ii) help with electrode targeting, for example in order to target stimulation to vertebral levels at which neuropathy is observed, (iii) enable a diagnostic tool to predict whether a given patient will respond well to SCS, noting that patients without doublets (and thus experiencing reduced postsynaptic activation) have been observed to gain greater benefit from SCS, and/or (iv) simply provide a variable which may be explored with patient input regarding their preferred therapy.

Should a given patient's circumstances reveal that PSDC fibres contribute to the pain relief, the CV of the elicited ECAP can be used to optimise the therapy. Here we assume knowledge of the 2 fibre types that exist and that the CV shows a ratio of activated fibres. So 45 m/s would be only PAs, 80+ m/s would be only PSDC, and anything in between would be a mix of PAs and PSDCs.

Should a given patient's circumstances reveal that PSDC fibres do not contribute to pain relief, it may be preferable to recruit as few PSDC fibres as possible. Therefore, seeking a stimulation configuration where PSDC activation is minimised may optimise therapy. The aim would be to use CV measurements to detect which stimulation paradigm is best. Minimising the CV may for example optimise therapy.

In yet other circumstances it may be investigated whether pain relief is obtained by desynchronization of PA and PSDC activation. In such cases it may be that having a mix of activation is optimal, so having a CV of about 60 m/s would be optimal and this would mean that as many PAs as PSDC fibres are activated at the same time. This is a very “unnatural” firing pattern as physiologically, PSDC fibres are always activated synaptically, not axonally like in SCS.

It is further noted that the above insights can be used to modulate perception of stimulation. Without intending to be limited by theory it would seem that PSDC fibres carry the bulk of the information to the brain, given that most PAs terminate before reaching the DC nuclei. Therefore, it would follow that having fewer PSDCs activated (either synaptically or axonally) would reduce the overall perceived amplitude of the stimulation. Desynchronisation of PAs and PSDCs through axonal activation of both through SCS is also very likely to lead to non-normal sensations which may or may not be rejected by the brain and could help reducing or eliminating the paraesthesia sensation in the patient.

Noting that pre-synaptic neurons are readily activated, whereas postsynaptic neurons undergo a synaptically evoked action potential only if the neurotransmitter is strong enough due to their operation via temporal and spatial summation, the present invention advantageously provides an ability to separately assess presynaptic activation as compared to postsynaptic activation. Further noting that activation of postsynaptic fibres can occur either axonally or synaptically, with measurable differences arising in the observed neural response in each case, the present invention further recognises that this presents an opportunity to separately assess synaptic activation of postsynaptic fibres as compared to axonal activation of postsynaptic fibres. Depending on a relevant disease state which may be present, or a therapeutic mode of activation which is clinically desired, the relative levels of activation of presynaptic neurons vs. postsynaptic neurons, and the relative levels of synaptic vs. axonal activation of postsynaptic fibres, may each thus provide an important diagnostic tool and/or therapy optimisation tool.

FIG. 10 is a neural schematic generally illustrating a neural pathway comprising two distinct sections. In the first section 1010 there exist only primary fibres 1030. The primary fibres 1030 branch so that one branch continues into the second section 1020 of the pathway, while the other branch extends to synapse 1040 and synapses onto projection neurons 1050. In the second section 1020, the projection neurons 1050 send their axons along the same pathway as the first branch of the primary fibres. The conduction velocities of the primary and secondary fibres can be different or may be the same. The synaptic process at 1040 can be monosynaptic or polysynaptic. The connectivity is also open, for example a scenario could be that only one primary fibre connects to one secondary fibre. But it could be that multiple primary fibres connect to a single secondary neuron (or vice versa). FIG. 10 facilitates description of a number of further examples of the present invention. These following examples relate to dorsal column stimulation where the primary fibres are primary sensory fibres (low threshold mechanoreceptor neurons whose axons are of the A-beta type), and the secondary fibres are postsynaptic dorsal column (PSDC) fibres. These PSDC neurons receive both monosynaptic and polysynaptic inputs from the primary afferents.

Example 1: This example seeks to identify whether stimulation is applied in section 1010 or in section 1020. The applied stimulation needs to be strong enough to elicit secondary fibre activation from primary afferent activation. The CV of each fibre type is not important here. Start with a sweep of the pathway by applying stimulation on one electrode contact and observing the neural response further up the pathway (rostrally). Repeat for all electrodes to be covered by the sweep. If the stimulation was applied in section 1020, a first type of response will be observed, which in the case of a single implanted device of limited size will mostly be a single ECAP, but if the recording is obtained from electrodes very far away and the conduction velocities of the primary and secondary fibres are distinctly different then the observed response will tend to exhibit temporally distinct peaks. On the other hand, if the stimulation was applied in section 1010, a second type of response will be observed, in the form of a doublet. A doublet refers to a more complex neural response made up of first the primary afferent ECAP, followed by the ECAP generated by the secondary fibres, after the synaptic delay. The time delay between the two ECAPs provides further information as to whether monosynaptic or polysynaptic transmission has occurred. Based on the obtained knowledge as to whether the stimulation was applied in section 1010 or section 1020, the stimulation location and/or stimulation parameters can be revised. This may include intraoperative targeting and surgical positioning of the electrodes, and/or selection of other electrodes to use for stimulation.

Example 2: To investigate the state of a synapse. A premise here is that it is known that stimulation is being applied in section 1010 and that recordings are being obtained in section 1020. This may be determined as per Example 1. Place the stimulation in section 1010, and monitor the response in section 1020. Should it be observed that the neural recording contains two ECAPs separated by a synaptic delay (i.e. “doublets”), then it can be concluded that the stimulation has synaptically activated the secondary fibres, i.e. by activating the primary fibres and having them synaptically activate the secondary fibres. Seeing doublets means that the synapse fired. If this is expected for the pathway in question then it can be concluded that the pathway is behaving “normally”. If stimulation is applied at a reduced intensity which is not expected to be strong enough to get the secondary neurons to fire, then an observation of doublets can enable a conclusion that the synapse is in a sensitised state. Alternatively, if stimulation is applied at a high intensity at which the secondary neurons are expected to fire, then an absence of doublets can enable a conclusion that the synapse is in a desensitised state. This knowledge can be used for diagnostic purposes (e.g. is my patient sensitised/desensitised), and for continuous monitoring and adaptation of targeting, stimulus parameter selection etc. This knowledge can additionally or alternatively be used as a proxy measure for drug intake, for example taking inhibitory drugs might cause depression of that synapse. Knowledge of the state of the synapse might be used as a proxy measure for general depression/sensitisation of the dorsal horn in particular. This could be used as a tool for diagnosis of neuropathic pain, and help targeting and optimisation, for example a patient that has a unilateral condition would have different responses based on whether the fibres stimulated are on the left or right, and so this knowledge can be used to make sure the right ones fibres are stimulated.

Example 3: Targeting and stimulus parameter optimisation in section 1020. This assumes that the conduction velocity distribution of the primary and secondary fibres are distinct or at least different enough to be differentiated. Stimulating in Section 1020, knowing that the two populations (1030 and 1050) coexist there, the conduction velocity measurement in either direction of the pathway will indicate if the stimulation recruits mainly primary fibres 1030, mainly secondary fibres 1050, or a reasonable mix of the two. Further, recording in section 1010 should only have the primary fibre 1030 response and can help identify the source of the ECAP further. Note that stimulating farther away in the orthodromic direction (to the right in FIG. 10) will produce recordings from electrodes in section 1020 in which the ECAP is split into two temporal parts due to the differing conduction velocity of 1020 as compared to 1050. Recordings from electrodes in section 1010 will exhibit a fall-off of the activity upon secondary fibres 1050 because they start at the synapse 1040. In the special case of dorsal column stimulation, it is known that the secondary fibres 1050 conduct much faster (approx. 90 m/s) than the primary fibres 1030 (approx. 45 m/s), so that if an observed ECAP has a conduction velocity around the same value as is expected for the secondary fibres 1050 then it can be concluded that the applied stimulus mainly activated PSDC fibres by way of axonal activation and not by synaptic activation. Without intending to be limited by theory, it is noted that activation of the PSDC fibres are not considered to contribute to pain relief, and so this observation allows a conclusion that the stimulation paradigm used is delivering sub-optimal therapy, in terms of both sub-optimal pain relief as well as sub-optimal power consumption. On the other hand an observed conduction velocity of 45 m/s or so would indicate that mostly primary afferents and few PSDC fibres are being activated as is desired for DC stimulation for pain relief. A conduction between 45 m/s and 90 m/s can be taken to indicate that a mix of primary fibres 1030 and PSDC fibres 1050 are being stimulated.

It is to be appreciated that post-synaptic neurons may be activated either directly by applied electrical stimulation, or by natural synapsing from pre-synaptic neurons. Thus the terms cross-synaptic, post-synaptic and the like are intended herein to encompass neural activity arising on post-synaptic neurons, however evoked.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not limiting or restrictive.

1. A neurostimulation system comprising:
at least one stimulation electrode configured to deliver an electrical stimulus to neural tissue;
at least one measurement electrode configured to record a neural recording of a response of the neural tissue to the stimulus; and
a processor configured to assess the neural recording to produce a measure of postsynaptic activation.

2. The system of claim 1 wherein the processor is further configured to assess both primary activation and postsynaptic activation.

3. The system of claim 1 wherein the processor is further configured to assess both axonal activation of postsynaptic fibres and synaptic activation of postsynaptic fibres.

4. The system of claim 1, comprising an implantable neurostimulator.

5. The system of claim 1 wherein the processor is further configured to assess the neural recording for additional lobes.

6. The system of claim 5 wherein the processor is further configured to use a presence or strength of additional lobes as a measure of the presence or strength of synaptic activation of postsynaptic fibres.

7. The system of claim 6 wherein the processor is further configured to compare the measure of the presence or strength of synaptic activation of postsynaptic fibres to the electrical stimulus, to determine a state of an intervening synapse.

8. The system of claim 6 wherein the processor is further configured to compare the measure of the presence or strength of synaptic activation of postsynaptic fibres to a presence or strength of primary activation, to determine a state of an intervening synapse.

9. The system of claim 1 wherein the processor is further configured to obtain a plurality of neural recordings of a response of the neural tissue to the respective stimulus from a respective plurality of measurement electrodes positioned along a fibre tract of the neural tissue, and wherein the processor is further configured to determine from the plurality of neural recordings a conduction velocity of at least one component of the response of the neural tissue.

10. The system of claim 9 wherein the processor is further configured to determine whether the response of the neural tissue comprises at least two components conducting at different conduction velocities and originating simultaneously, and if so the processor being configured to output an indication that the neural response involved axonal activation of two different fibre types.

11. The system of claim 9 wherein the processor is further configured to determine whether the response of the neural tissue comprises at least two components originating at distinct times consistent with synaptic delay, and if so the processor being configured to output an indication that the neural response involved synaptic activation of a postsynaptic fibre.

12. The system of claim 9 wherein the processor is further configured to compare a presence or strength of synaptic activation of postsynaptic fibres to the electrical stimulus, to determine a state of an intervening synapse.

13. The system of claim 9 wherein the processor is further configured to compare a presence or strength of synaptic activation of postsynaptic fibres to a presence or strength of primary activation, to determine a state of an intervening synapse.

14. The system of claim 7, further comprising a diagnostic module configured to receive an indicium as to the state of the intervening synapse and configured to produce therefrom a prediction as to whether the patient will benefit from neuromodulation.

15. The system of claim 1 wherein the processor is further configured to use the measure of postsynaptic activation to revise at least one stimulation parameter for a subsequent stimulus.

16. A method of neurostimulation, the method comprising:
delivering an electrical stimulus to neural tissue using at least one stimulation electrode;
obtaining a neural recording of a response of the neural tissue to the stimulus using at least one measurement electrode, and
assessing the neural recording to produce a measure of postsynaptic activation.

17. A non-transitory computer readable medium for neurostimulation, comprising instructions which, when executed by one or more processors, causes performance of the following:
delivering an electrical stimulus to neural tissue using at least one stimulation electrode;
obtaining a neural recording of a response of the neural tissue to the stimulus using at least one measurement electrode, and
assessing the neural recording to produce a measure of postsynaptic activation.

18. The system of claim 8, further comprising a diagnostic module configured to receive an indicium as to the state of the intervening synapse and configured to produce therefrom a prediction as to whether the patient will benefit from neuromodulation.

19. The system of claim 12, further comprising a diagnostic module configured to receive an indicium as to the state of the intervening synapse and configured to produce therefrom a prediction as to whether the patient will benefit from neuromodulation.

20. The system of claim 13, further comprising a diagnostic module configured to receive an indicium as to the state of the intervening synapse and configured to produce therefrom a prediction as to whether the patient will benefit from neuromodulation.
